(19) United States
(12) Patent Application Publication
(10) Pub. No.: US 2017/0191015 A1
(43) Pub. Date: Jul. 6, 2017

(54) GAS HEATING APPARATUS FOR DISPOSABLE BIOREACTOR

(71) Applicant: Sarfaraz K. Niazi, Deerfield, IL (US)

(72) Inventor: Sarfaraz K. Niazi, Deerfield, IL (US)

(73) Assignee: THERAPEUTIC PROTEINS INTERNATIONAL, LLC, Chicago, IL (US)

(21) Appl. No.: 14/987,426

(22) Filed: Jan. 4, 2016

(51) Int. Cl. C12M 1/00; C12M 1/34; C12M 3/06; C12M 1/02

(52) U.S. Cl. CPC C12M 23/28; C12M 41/20; C12M 41/26; C12M 41/34; C12M 23/14; C12M 27/16

(57) ABSTRACT

The present invention relates to an apparatus comprising a single-use circular bag having a sealed edge, capable of holding a nutrient media and designed to deliver heated or cooled air/gas into the media thereby aerating and maintaining the appropriate temperature for growth of a cell culture. In addition mixing is provided by the use of acoustic radiation devices located below a support structure.

### BACKGROUND

Bioreactors include containers used for fermentation, enzymatic reactions, cell culture, tissue engineering, and food production, as well as in the manufacture of biologicals, chemicals, biopharmaceuticals, microorganisms, plant metabolites, and the like. Bioreactors vary in size from benchtop fermenters to stand-alone units of various sizes. Small-scale bioreactors have also been developed which comprise pre-sterilized, disposable flexible bags configured to hold cell culture media.

As cell fermentation processes are highly sensitive to temperature variations, bioreactor systems require temperature-control mechanisms to maintain uniformity and stability of temperature throughout the bioreactor medium. Control mechanisms exist which comprise a heating blanket configured to surround a bioreactor bag. Such a heating blanket may comprise, for example, a silicon rubber blanket with wires running through it. These resistive heat blankets, however, are capable of heating the bioreactor medium but cannot cool the medium.

One method of providing both heating and cooling capability in a disposable bioreactor system is to provide a double-walled rigid vessel to support the bioreactor bag. The double walls of the vessel are filled with a fluid, such as water, which is circulated around the bag and pumped through an external heating or cooling device. Double-walled rigid vessels such as these, however, can be extremely expensive.

Mixing has been accomplished in the bioreactor using impeller devices, or, it has been accomplished by rocking of the container the bioreactor back and forth. For example, as shown in U.S. Pat. No. 6,544,788, to Singh, a disposable bioreactor is disclosed which accomplishes mixing by such a back and forth motion/process. This process is limited and cannot be utilized in a quick and efficient manner. Specifically, the rocking motion is limited to a low number of back and forth movements so as not to stress the bag and system. It also limits the size of the container. The present invention provides a solution to these problems.

### SUMMARY OF THE INVENTION

The present invention relates to an apparatus comprising a single-use circular bag having a sealed edge, capable of holding a nutrient media and designed to deliver heated or cooled air/gas into the media thereby aerating and maintaining the appropriate temperature for growth of a cell culture. The temperature controlled air/gas is delivered through an inlet at the top or bottom surface of the bag into a series of 5-6 porous pouches arranged on the bottom surface of the bag. Once the cell culture has produced a desired recombinant product, resin can be pumped into the porous pouches to allow capture of the product. The bag can contain a temperature sensor for monitoring the temperature of the medium. The air inlet will have a temperature controller for increasing or decreasing the temperature of the gas based on the temperature measured by the sensor.

The sealed edge may contain holes, grommets or other means for attaching the bag to a supportive platform. The platform is comprised of a grid or mesh composed of metal, glass or rigid polymer.

Optionally, the present invention includes the use of acoustic radiation to mix the cell culture. The source of acoustic radiation is attached below the perforated surface of the support platform. The acoustic radiation source is capable of producing acoustic waves from 2-300 Hz. The acoustic radiation source may be any device capable of delivering the acoustic waves of the proper frequency. The frequency of the acoustic waves may be altered during the various stages of the process, such as during cultivation, washing and/or elution stages.

In addition, the bioreactor may optionally be connected to a condenser to condense liquid particles in the exhaust gas. The condenser has a cooling surface capable of reaching temperatures sufficient to freeze the liquid particles.

The present invention provides a method of cultivating and harvesting proteins comprising the bioreactor described above; providing a sufficient quantity of nutrient media and biological culture to produce a target protein; heating the nutrient media and biological culture by starting flow of heated gas; optionally providing acoustic radiation to aid in mixing of the media; adjusting reaction conditions as needed for optimal growth and expression of proteins; closing the gas/liquid inlet to stop flow of gas and opening the gas/liquid inlet to introduce a binding resin after the cycle of upstream expression is complete; closing the gas/liquid port and opening the gal/liquid port gas inlet; adjusting the temperature of inlet gas to a suitable temperature optimal for binding of protein to the resin; draining the nutrient media and biological culture upon completion of binding of proteins to resin through liquid outlet; washing the resin in the tubular porous pouch with a washing liquid entered through the gas/liquid inlet; and eluting the protein by washing with an eluting medium or buffer and collecting the protein solution through the liquid outlet.

The heated gas may be a single gas or a mixture of gases such as oxygen (O2) or carbon dioxide (CO2) combined with a noble or inert gas such as nitrogen (N2), helium (He), neon (Ne), argon (Ar), krypton (Kr), xenon (Xe), or radon (Rn), or a mixture thereof. The composition of the mixture of gas may be altered during the cultivation of biological entities.

### BRIEF DESCRIPTION OF THE DRAWING

FIG. 1 is a top view of the bottom of the bioreactor depicting the porous pouches for heated/cooled air.

FIG. 2 is a side view of the apparatus depicting the bioreactor and the porous pouches where the air is introduced from the top of the bioreactor.

FIG. 3 is a side view of the apparatus depicting the bioreactor and the porous pouches where the air is introduced from the bottom.

FIG. 4 is a top view depicting the support for the bioreactor and speakers for delivering the acoustic radiation.

FIG. 5 is a perspective view showing one possible arrangement of the speakers positioned below the support and a controller for controlling the frequency and volume of the acoustic radiation.

### DETAILED DESCRIPTION OF THE INVENTION

Traditional recombinant protein manufacturing involves growing genetically modified organisms or cells in a culture media, harvesting the target protein from the rest of the contents of the nutrient media including recombinant cells or organisms and then purifying the target protein using column chromatography.

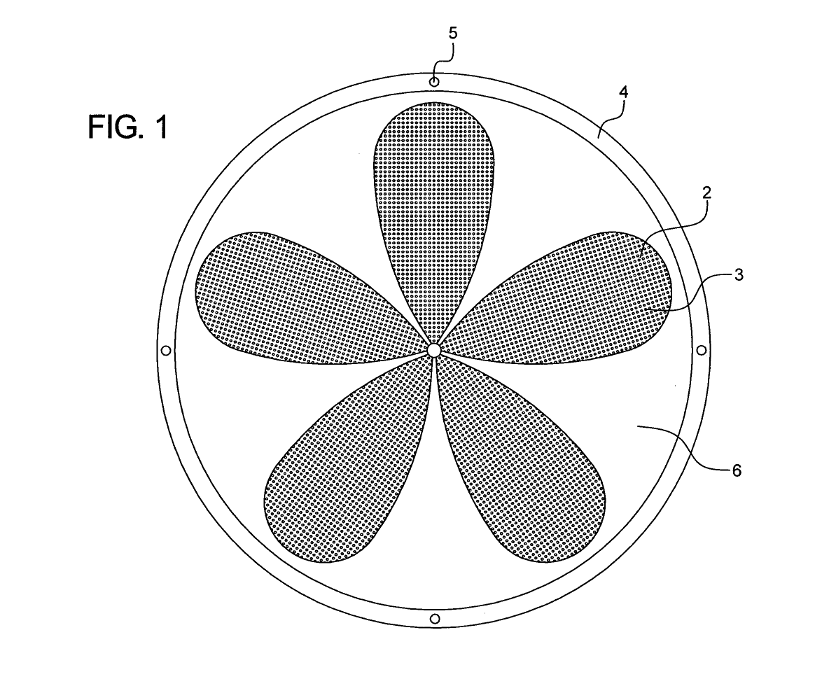

The present invention comprises an apparatus used to deliver heated air to the biological culture through a system of porous pouches located on the bottom surface of a circular bioreactor. FIG. 1 shows the arrangement of the porous pouches comprised of a biocompatible material and having pores sufficiently small to generate air bubbles to aerate the media and heat the culture without causing damage to the cells. If the porous pouches are also used to retain resin or chromatography material for capturing protein, the pores are of a size that does not allow the resin to escape from the pouch, e.g., between generally 50-300 microns. This size range also prevents the biological culture cells from entering the pouches.

The bottom inside surface of the bioreactor (1) is depicted in FIG. 1. A series of four to six equally spaced pouches (2) are attached or formed as part of the bottom surface (6) of the bioreactor. Each pouch comprises numerous pores (3) ranging in size from 1 micron to 300 microns. The size is dependent on the amount of air to be delivered and whether a resin will be injected into the pouches. The more air to be distributed the more pores and greater size. If resin is to be introduced, the pores must be smaller than the average size of the resin particles. The bottom surface also has an outer seam (4) with openings (5) to allow it to be fastened to a support.

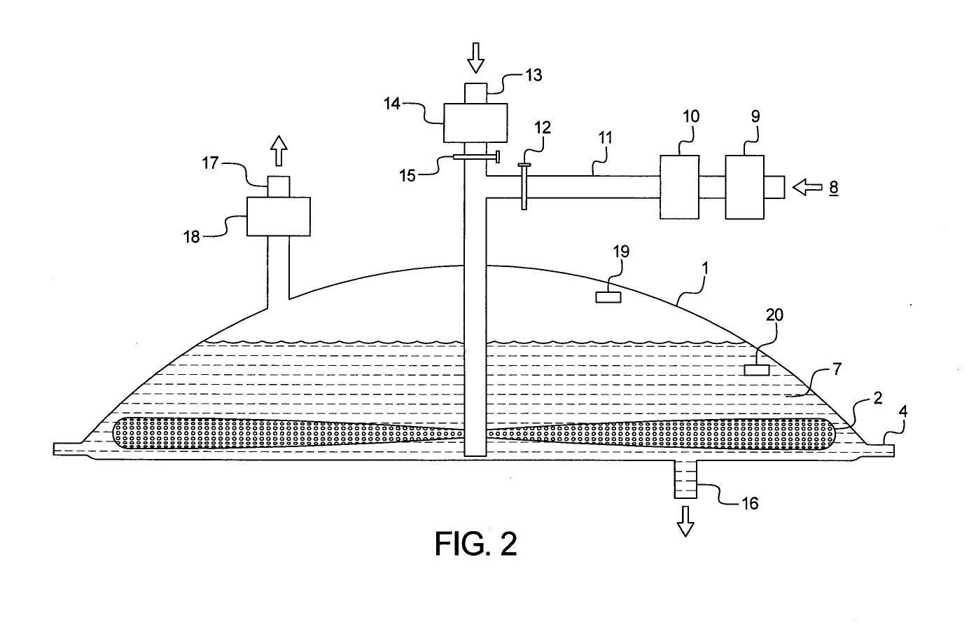

FIG. 2 depicts the side view of the bioreactor (1) showing the various ports including the apparatus used to deliver the heated/cooled air and liquid or resin. The port (8) to be attached to a source of gas comprises a heating or cooling element with controller that allows the operator to change the temperature of the gas entering the bioreactor chamber and a filter (10) to sterilize the gas before it enter the bioreactor. The tube leading from the port (11) can be closed or opened via the gas closure valve (12). This tube connects at a T-junction to the main tube (13) leading to the bioreactor chamber that is connected to the pouches (2). The chromatography material or resin can be introduced through this inlet. The inlet can optionally comprise a filter (14) and a liquid/resin closure valve (15). Liquid can be removed from the bioreactor via the liquid outlet port (16) and gas can be released from the bioreactor via a gas outlet port (17), which may comprise a filter (18) to trap any contaminants. The bioreactor may also contain sensors, such as a pressure sensor (19) and a temperature sensor (20).

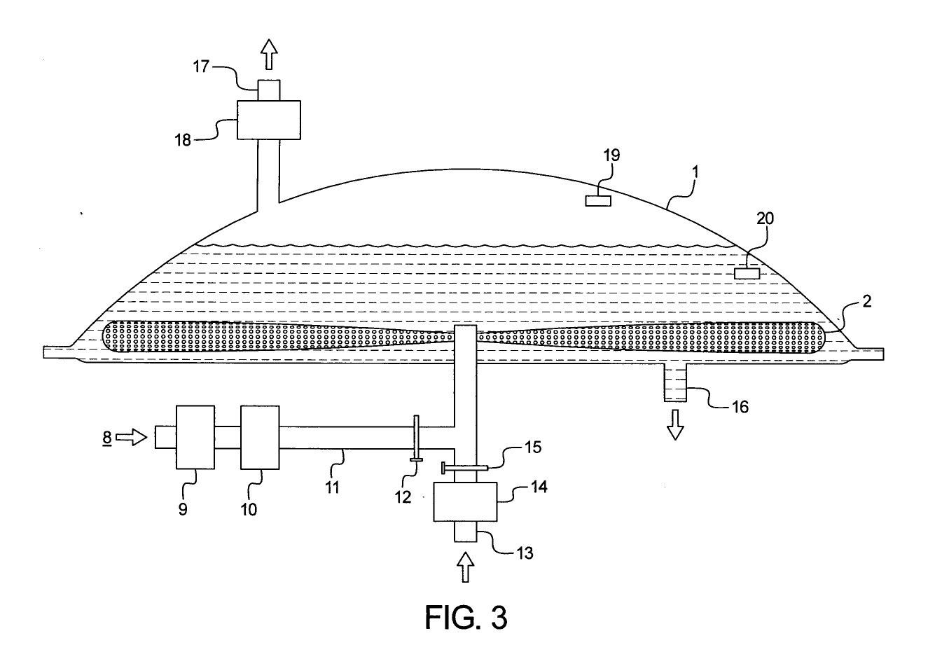

FIG. 3 depicts an alternative arrangement of the apparatus used to introduce air and liquid or resin from the bottom of the bioreactor.

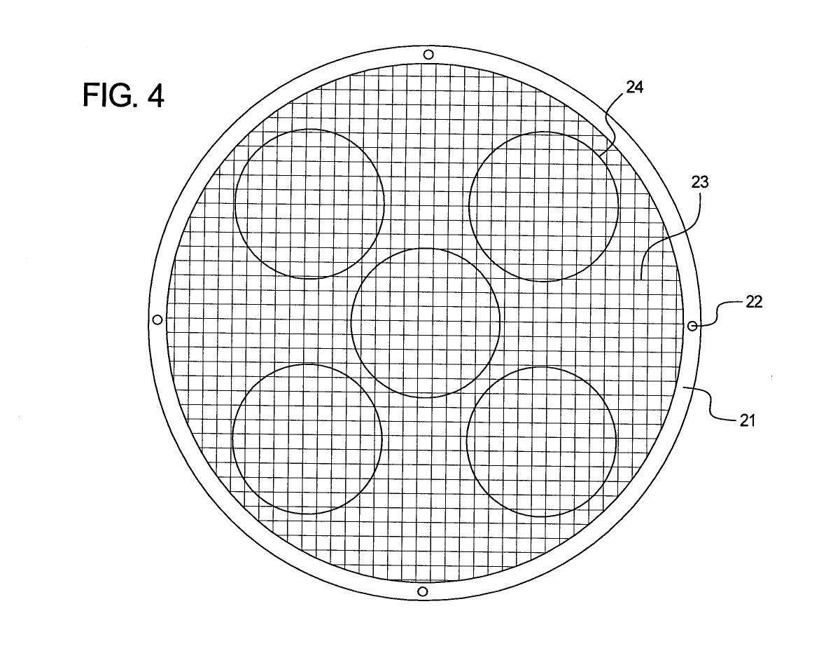

FIG. 4 depicts the support used for the bioreactor and the arrangement of devices for delivering the acoustic radiation, such as speakers. The rim of the support (21) matches the dimensions of the bioreactor bag to be placed on the support and the holes (22) correspond to the placement of holes in the bioreactor for attaching the bioreactor. The acoustic devices (24) are arranged below the support mesh (23).

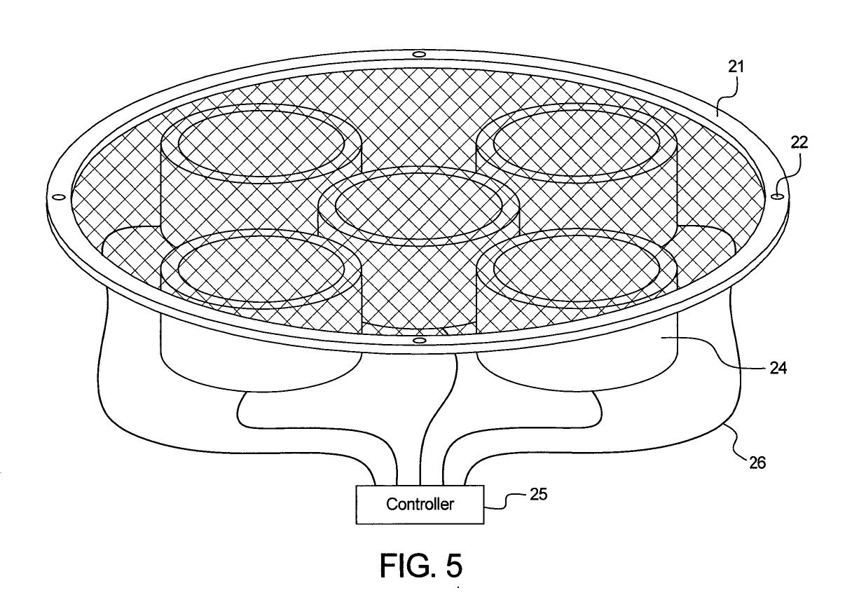

FIG. 5 depicts a side view of the support and acoustic devices. Connectors (26) for connecting the devices to a controller (25) are also depicted. The controller is for controlling the volume and frequency of the acoustic devices. It can also alternate the signal turning the devices on and off, such that the sound can be delivered through all devices or in a series circling around the bioreactor, such that it is capable of generating a wave.

The present invention includes the use of acoustic radiation to mix the cell culture. The source of acoustic radiation is attached below the perforated surface of the support platform and is capable of producing acoustic waves from 2-300 Hz. The acoustic radiation source may be any device capable of delivering the acoustic waves of the proper frequency. The frequency of the acoustic waves may be altered during the various stages of the process, such as during cultivation, washing and/or elution stages.

The present invention allows for more precise delivery of heating and cooling to the contents of the bioreactor. Temperature controlled gas is delivered to the bioreactor with equal distribution throughout the bioreactor through the pouches. The temperature sensor allows for precise measurement of temperature inside the bioreactor and the controller on the gas inlet controls the temperature of the gas entering to adjust rapidly to changes in temperature. The heated gas may be a single gas or a mixture of gases such as oxygen (O2) or carbon dioxide (CO2) combined with a noble or inert gas such as nitrogen (N2), helium (He), neon (Ne), argon (Ar), krypton (Kr), xenon (Xe), or radon (Rn), or a mixture thereof. The composition of the mixture of gas may be altered during the cultivation of biological entities.

These same pouches can be used to capture the protein produced in the bioreactor at the end of the production cycle without having to centrifuge the cells. Chromatography or resin material is introduced into the pouches and the protein is allowed to bind to the resin. Then the cells and spent media can be drained out through the liquid outlet.

An eluting liquid may be introduced into the apparatus, allowing it to mix and elute and then drain the eluting liquid with the released protein from the apparatus through the liquid outlet. Because the transfer of liquid is achieved by gravity flow, there is no strain on protein or cells that may come from the use of peristaltic pumps in the transfer of nutrient media to and from the apparatus. The apparatus is disposable, and the bioreactor is then disposed of without ever having to open the bioreactor. This reduces the possibility of contamination of the product.

The present invention describes a method of keeping the chromatography resin binding the protein separate from the nutrient media inside a bioreactor and thus allowing separation of wasted nutrient media and cells by simply draining the bioreactor. This eliminates at least three steps in downstream processing, viz., filtration of culture broth to remove cells, cross-flow filtration to reduce the volume of broth and finally loading of protein solution onto a separation column.

Examples of resin that may be used in the present invention include, but are not limited to: Dual Affinity Polypeptide technology platform; Protein A; Protein G; stimuli responsive polymers enable complexation and manipulation of proteins; mixed mode sorbents; ion exchange media; hydrophobic charge induction chromatography, such as MEP, and Q and S HyperCel; Monoliths, such as Convective Interaction Media monolithic columns; simulated moving beds, such as BioSMB; single domain camel-derived (camelid) antibodies to IgG, such as CaptureSelect; inorganic ligands, including synthetic dyes, such as Mabsorbent A1P and A2P; Expanded bed adsorption chromatography systems, such as the Rhobust platform; ultra-durable zirconia oxide-bound affinity ligand chromatography media; Fc-receptor mimetic ligand; ADSEPT (ADvanced SEParation Technology); membrane affinity purification system; custom-designed peptidic ligands for affinity chromatography; protein A- and G-coated magnetic beads; affinity purification methods based on expression of proteins or MAbs as fusion proteins with removable portion (tag) having affinity for chromatography media, such as histidine tags; protein A alternatives in development; plug-and-play solutions with disposable components; affinity chromatography media; lectin chromatography media; and immunoaffinity chromatography media.

The present invention allows for the use of a mixed-bed chromatography resin that may contain an ionic chromatography resin, a hydrophobic chromatography resin and/or an affinity chromatography resin all used together to optimize the efficiency of harvesting. It is well established that the use of ionic chromatography resins does not allow complete capture of proteins because of the logarithmic nature of ionization. However, a combination of chromatography resins used in the present invention allows for a more complete recovery of target proteins. Since the purpose of reaction at the chromatography resin-protein complexation stage is to harvest and not purify the protein, the calculations like chromatography plates for purification are not important and neither is the particle size o the chromatography resin allowing use of the cheapest chromatography resin available. Any lack of efficiency in capturing proteins can be readily adjusted by increasing the quantity of chromatography resin. The chromatography resin can be used repeatedly after washing of the proteins and sanitizing the chromatography resin.

Examples of cells that can be used in the operation of the bioreactor, include, but are not limited to: Chinese hamster ovary (CHO), mouse myeloma cells, M0035 (NSO cell line), hybridomas (e.g., B-lymphocyte cells fused with myeloma tumor cells), baby hamster kidney (BHK), monkey COS, African green monkey kidney epithelial (VERO), mouse embryo fibroblasts (NIH-3T3), mouse connective tissue fibroblasts (L929), bovine aorta endothelial (BAE-1), mouse myeloma lymphoblastoid-like (NSO), mouse B-cell lymphoma lymphoblastoid (WEHI 231), mouse lymphoma lymphoblastoid (YAC 1), mouse fibroblast (LS), hepatic mouse (e.g., MC/9, NCTC clone 1469), and hepatic rat cells (e.g., ARL-6, BRL3A, H4S, Phi 1 (from Fu5 cells)). Human cells include retinal cells (PER-C6), embryonic kidney cells (HEK-293), lung fibroblasts (MRC-5), cervix epithelial cells (HELA), diploid fibroblasts (WI38), kidney epithelial cells (HEK 293), liver epithelial cells (HEPG2), lymphoma lymphoblastoid cells (Namalwa), leukemia lymphoblastoid-like cells (HL60), myeloma lymphoblastoid cells (U 266B1), neuroblastoma neuroblasts (SH-SY5Y), diploid cell strain cells (e.g., propagation of poliomyelitis virus), pancreatic islet cells, embryonic stem cells (hES), human mesenchymal stem cells (MSCs, which can be differentiated to osteogenic, chondrogenic, tenogenic, myogenic, adipogenic, and marrow stromal lineages, for example), human neural stem cells (NSC), human histiocytic lymphoma lymphoblastoid cells (U937), and human hepatic cells such as WRL68 (from embryo cells), PLC/PRF/5 (i.e., containing hepatitis B sequences), Hep3B (i.e., producing plasma proteins: fibrinogen, alpha-fetoprotein, transferrin, albumin, complement C3 and/or alpha-2-macroglobulin), and HepG2 (i.e., producing plasma proteins: prothrombin, antithrombin III, alpha-fetoprotein, complement C3, and/or fibrinogen).

Cells from insects (e.g., baculovirus and Spodoptera frugiperda ovary (Sf21 cells produce Sf9 line)) and cells from plants or food, may also be cultured in accordance with the invention. Cells from sources such as rice (e.g., Oryza sativa, Oryza sativa cv Bengal callus culture, and Oryza sativa cv Taipei 309), soybean (e.g., Glycine max cv Williams 82), tomato (Lycopersicum esculentum cv Seokwang), and tobacco leaves (e.g., Agrobacterium tumefaciens including Bright Yellow 2 (BY-2), Nicotiana tabacum cv NT-1, N. tabacum cv BY-2, and N. tabacum cv Petite Havana SR-1) are illustrative examples.

Bacteria, fungi, or yeast may also be cultured in accordance with the invention. Illustrative bacteria include Salmonella, Escherichia coli, Vibrio cholerae, Bacillus subtilis, Streptomyces, Pseudomonas fluorescens, Pseudomonas putida, Pseudomonas sp, Rhodococcus sp, Streptomyces sp, and Alcaligenes sp. Fungal cells can be cultured from species such as Aspergillus niger and Trichoderma reesei, and yeast cells can include cells from Hansenula polymorpha, Pichia pastoris, Saccharomyces cerevisiae, S. cerevisiae crossed with S. bayanus, S. cerevisiae crossed with LAC4 and LAC1-2 genes from K. lactis, S. cerevisiae crossed with Aspergillus shirousamii, Bacillus subtilis, Saccharomyces diastasicus, Schwanniomyces occidentalis, S. cerevisiae with genes from Pichia stipitis, and Schizosaccharomyces pombe.

A variety of different products may also be produced in accordance with the invention. Illustrative products include proteins (e.g., antibodies and enzymes), vaccines, viral products, hormones, immunoregulators, metabolites, fatty acids, vitamins, drugs, antibiotics, cells, and tissues. Non-limiting examples of proteins include human tissue plasminogen activators (tPA), blood coagulation factors, growth factors (e.g., cytokines, including interferons and chemokines), adhesion molecules, Bcl-2 family of proteins, polyhedrin proteins, human serum albumin, scFv antibody fragment, human erythropoietin, mouse monoclonal heavy chain 7, mouse IgG.sub.2b/k, mouse IgG1, heavy chain mAb, Bryondin 1, human interleukin-2, human interleukin-4, ricin, human .alpha.1-antitrypisin, biscFv antibody fragment, immunoglobulins, human granulocyte, stimulating factor (hGM-CSF), hepatitis B surface antigen (HBsAg), human lysozyme, IL-12, and mAb against HBsAg. Examples of plasma proteins include fibrinogen, alpha-fetoprotein, transferrin, albumin, complement C3 and alpha-2-macroglobulin, prothrombin, antithrombin III, alpha-fetoprotein, complement C3 and fibrinogen, insulin, hepatitis B surface antigen, urate oxidase, glucagon, granulocyte-macrophage colony stimulating factor, hirudin/desirudin, angiostatin, elastase inhibitor, endostatin, epidermal growth factor analog, insulin-like growth factor-1, kallikrein inhibitor, .alpha.1-antitrypsin, tumor necrosis factor, collagen protein domains (but not whole collagen glycoproteins), proteins without metabolic byproducts, human albumin, bovine albumin, thrombomodulin, transferrin, factor VIII for hemophilia A (i.e., from CHO or BHK cells), factor VIIa (i.e., from BHK), factor IX for hemophilia B (i.e., from CHO), human-secreted alkaline phosphatase, aprotinin, histamine, leukotrienes, IgE receptors, N-acetylglucosaminyltransferase-III, and antihemophilic factor VIII.

Enzymes may be produced from a variety of sources using the invention. Non-limiting examples of such enzymes include YepACT-AMY-ACT-X24 hybrid enzyme from yeast, Aspergillus oryzae .alpha.-amylase, xylanases, urokinase, tissue plasminogen activator (rt-PA), bovine chymosin, glucocerebrosidase (therapeutic enzyme for Gaucher's disease, from CHO), lactase, trypsin, aprotinin, human lactoferrin, lysozyme, and oleosines.

Vaccines also may be produced using the invention. Non-limiting examples include vaccines for prostate cancer, human papilloma virus, viral influenza, trivalent hemagglutinin influenza, AIDS, HIV, malaria, anthrax, bacterial meningitis, chicken pox, cholera, diphtheria, haemophilus influenza type B, hepatitis A, hepatitis B, pertussis, plague, pneumococcal pneumonia, polio, rabies, human-rabies, tetanus, typhoid fever, yellow fever, veterinary-FMD, New Castle's Disease, foot and mouth disease, DNA, Venezuelan equine encephalitis virus, cancer (colon cancer) vaccines (i.e., prophylactic or therapeutic), MMR (measles, mumps, rubella), yellow fever, Haemophilus influenzae (Hib), DTP (diphtheria and tetanus vaccines, with pertussis subunit), vaccines linked to polysaccharides (e.g., Hib, Neisseria meningococcus), Staphylococcus pneumoniae, nicotine, multiple sclerosis, bovine spongiform encephalopathy (mad cow disease), IgG1 (phosphonate ester), IgM (neuropeptide hapten), SIgA/G (Streptococcus mutans adhesin), scFv-bryodin 1 immunotoxin (CD-40), IgG (HSV), LSC (HSV), Norwalk virus, human cytomegalovirus, rotavirus, respiratory syncytial virus F, insulin-dependent autoimmune mellitus diabetes, diarrhea, rhinovirus, herpes simplex virus, and personalized cancer vaccines, e.g., for lymphoma treatment (i.e., in injectable, oral, or edible forms). Recombinant subunit vaccines also may be produced, such as hepatitis B virus envelope protein, rabies virus glycoprotein, E. coli heat labile enterotoxin, Norwalk virus capsid protein, diabetes autoantigen, cholera toxin B subunit, cholera toxin B an dA2 subunits, rotavirus enterotoxin and enterotoxigenic E. coli, fimbrial antigen fusion, and porcine transmissible gastroenteritis virus glycoprotein S.

Viral products also may be produced. Non-limiting examples of viral products include sindbis, VSV, oncoma, hepatitis A, channel cat fish virus, RSV, corona virus, FMDV, rabies, polio, reo virus, measles, and mumps.

Hormones also may be produced using the invention. Non-limiting examples of hormones include growth hormone (e.g., human growth hormone (hGH) and bovine growth hormone), growth factors, beta and gamma interferon, vascular endothelial growth factor (VEGF), somatostatin, platelet-derived growth factor (PDGF), follicle stimulating hormone (FSH), luteinizing hormone, human chorionic hormone, and erythropoietin.

Immunoregulators also may be produced. Non-limiting examples of immunoregulators include interferons (e.g., beta-interferon (for multiple sclerosis), alpha-interferon, and gamma-interferon) and interleukins (such as IL-2).

Metabolites (e.g., shikonin and paclitaxel) and fatty acids (i.e., including straight-chain (e.g., adipic acid, Azelaic acid, 2-hydroxy acids), branched-chain (e.g., 10-methyl octadecanoic acid and retinoic acid), ring-including fatty acids (e.g., coronaric acid and lipoic acid), and complex fatty acids (e.g., fatty acyl-CoA)) also may be produced.

The containers useful in the various embodiments of the invention may be of any size suitable for containing a liquid. For example, the container may have a volume between 1-40 L, 40-100 L, 100-200 L, 200-300 L, 300-500 L, 500-750 L, 750-1,000 L, 1,000-2,000 L, 2,000-5,000 L, or 5,000-10,000 L. In some instances, the container has a volume greater than 1 L, or in other instances, greater than 10 L, 20 L, 40 L, 100 L, 200 L, 500 L, or 1,000 L. Volumes greater than 10,000 L are also possible. Preferably, the container volume will range between about 1 L and 1000 L, and more preferably between about 5 L and 500 L, and even more preferably between 5 L and 200 L.

The components of the bioreactors and other devices described herein, which come into contact with the culture medium or products provided thereby, desirably comprise biocompatible materials, more desirably biocompatible polymers, and are preferably the materials can be sterilized.

It should also be understood that many of the components described herein also are desirably flexible, e.g., the bioreactor desirably comprises a flexible biocompatible polymer (such as a collapsible bag), with the conduits also desirably comprising such biocompatible polymers. The flexible material is further desirably one that is USP Class VI certified, e.g., silicone, polycarbonate, polyethylene, and polypropylene. Non-limiting examples of flexible materials include polymers such as polyethylene (e.g., linear low density polyethylene and ultra low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. If desired, portions of the flexible container may comprise a substantially rigid material such as a rigid polymer (e.g., high density polyethylene), metal, and/or glass.

The bioreactor may have any thickness suitable for retaining the culture medium within, and may be designed to have a certain resistance to puncturing during operation or while being handled. For example, the walls of the bioreactor may have a total thickness of less than or equal to 250 mils (1 mil is 25.4 micrometers), less than or equal to 200 mils, less than or equal to 100 mils, less than or equal to 70 mils (1 mil is 25.4 micrometers), less than or equal to 50 mils, less than or equal to 25 mils, less than or equal to 15 mils, less than or equal to 10 mils, less than or equal to 5 mils, or less than or equal to 3 mils, or combinations thereof. In certain embodiments, the bioreactor may include more than one layer of material that may be laminated together or otherwise attached to one another to impart certain properties to the bioreactor. For instance, one layer may be formed of a material that is substantially oxygen impermeable. Another layer may be formed of a material to impart strength to the bioreactor. Yet another layer may be included to impart chemical resistance to fluid that may be contained in the bioreactor.

The embodiments described above do not in any way comprise all embodiments that are possible using the present invention and one with ordinary skills in the art would find many more applications specific to a complex process or even in those processes where such needs might not be immediately apparent.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A bioreactor comprising:
a single-use circular bag with a top and a bottom, sealed edge, and capable of holding a nutrient media;
at least one gas/liquid inlet connected at the top surface;
at least one liquid outlet connected to the bottom surface;
a heating and cooling element connected to gas/liquid inlet;
at least one tubular porous pouch connected to the gas/liquid inlet and disposed inside the bag;
at least one temperature sensor disposed inside or outside of the bag; and
a controller to adjust the temperature of an inlet gas.

2. The bioreactor of claim 1, wherein the bag further comprises additional sensors.

3. The bioreactor of claim 2, wherein the sensors are selected from pH, pO2 or pCO2 measurement.

4. The bioreactor of claim 1, wherein the gas/liquid inlet is further comprises a mass flow controller to mix a plurality of gases.

5. The bioreactor of claim 1, wherein the tubular porous pouch is tufted.

6. The bioreactor of claim 1, further comprising a plurality of tubular porous pouches connected to the gas/liquid inlet.

7. The bioreactor of claim 1, wherein the tubular porous pouch is secured to the bottom inner surface of the bag.

8. The bioreactor of claim 1, wherein the pore size of porous pouch is 5-300 microns.

9. The bioreactor of claim 1, wherein the bag is lined with a layer of a polytetrafluoroethylene membrane.

10. The bioreactor of claim 1, wherein the sealed edge of the bag includes holes, grommets or other devices for holding the bag on a supporting base.

11. The bioreactor of claim 10, wherein the supporting base is a metal mesh, a perforated plastic sheet, or perforated glass.

12. The bioreactor of claim 11, further comprising at least one source of acoustic radiation attached below the perforated surface of the support.

13. The bioreactor of claim 12, wherein the acoustic radiation source is capable of producing acoustic waves from 2-300 Hz.

14. The bioreactor of claim 1, wherein the gas outlet is connected to a condenser to condense liquid particles.

15. The bioreactor of claim 14, wherein the condenser has a cooling surface capable of reaching temperatures sufficient to freeze the liquid particles.

16. A method of cultivating and harvesting proteins comprising:
providing the bioreactor of claim 1;
providing a sufficient quantity of nutrient media and biological culture to produce a target protein;
heating the nutrient media and biological culture by starting flow of heated gas;
starting the acoustic radiation;
adjusting reaction conditions as needed for optimal growth and expression of proteins;
closing the gas/liquid inlet to stop flow of gas and opening the gas/liquid inlet to introduce a binding resin after the cycle of upstream expression is complete;
closing the gas/liquid port liquid inlet and opening the gal/liquid port gas inlet;
adjusting the temperature of inlet gas to a suitable temperature optimal for binding of protein to the resin.
draining the nutrient media and biological culture upon completion of binding of proteins to resin through liquid outlet;
washing the resin in the tubular porous pouch with a washing liquid entered through the gas/liquid inlet;
eluting the protein by washing with an eluting medium and collecting the protein solution through liquid outlet.

17. The method of claim 16, wherein the temperature of gas is programmed to change during the cultivation of biological entities.

18. The method of claim 17, wherein the gas is a mixture of nutrient gases such as oxygen (O2) or carbon dioxide (CO2) and inert gases such as nitrogen (N2), helium (He), neon (Ne), argon (Ar), krypton (Kr), xenon (Xe), or radon (Rn), or a mixture thereof.

19. The method of claim 17, wherein the composition of the mixture of gas is altered during the cultivation of biological entities.

20. The method of claim 14, wherein the frequency of the acoustic waves is altered during the cultivation, washing and elution stages.
